(19) United States
(12) Patent Application Publication
(10) Pub. No.: US 2026/0208196 A1
(43) Pub. Date: Jul. 23, 2026

(54) RAPID DETECTION KIT FOR PEDIATRIC RESPIRATORY INFECTION

(71) Applicant: Baoding Hospital, Beijing Children's Hospital, Capital Medical University, Baoding (CN)

(72) Inventors: Adong SHEN, Baoding (CN); Jing BI, Baoding (CN); Tingting JIANG, Baoding (CN); He TANG, Baoding (CN)

(73) Assignee: Baoding Hospital, Beijing Children's Hospital, Capital Medical University, Baoding (CN)

(21) Appl. No.: 19/248,537

(22) Filed: Jun. 25, 2025

(30) Foreign Application Priority Data
Jan. 22, 2025 (CN) 202510100428.1

(51) Int. Cl. B01L 3/00

(52) U.S. Cl. CPC B01L 3/523; B01L 2200/0689; B01L 2300/042; B01L 2300/047

(57) ABSTRACT

The rapid detection kit includes a first sub kit for containing a rapid detection reagent for pediatric respiratory infection. The first sub kit is externally sleeved with a first protective sleeve, a top end of the first protective sleeve is integrally formed with a connecting ring, and a second sub kit is threadedly connected inside the connecting ring. The second sub kit internally contains the rapid detection reagent for pediatric respiratory infection, a cap is threadedly connected to the top end of the second sub kit, the second sub kit is externally sleeved with a second protective sleeve, and the bottom end of the second protective sleeve externally sleeves the connecting ring.

### CROSS-REFERENCE TO THE RELATED APPLICATIONS

This application is based upon and claims priority to Chinese Patent Application No. 202510100428.1, filed on Jan. 22, 2025, the entire contents of which are incorporated herein by reference.

### TECHNICAL FIELD

The present disclosure relates to the technical field of kits, and particularly to a rapid detection kit for pediatric respiratory infection.

### BACKGROUND

A respiratory infection is one of common diseases in the pediatric medical field, and the rapid and accurate detection on the respiratory infection is crucial for the diagnosis and treatment of the disease. Traditional detection methods often take a long time and are complicated in operation, which are not conducive to timely diagnosis and treatment of pediatric patients. Therefore, it is particularly important to develop a fast, easy and accurate detection kit for pediatric respiratory infection.

Conventional detection kits for pediatric respiratory infection mostly use single-tube containers to load detection reagents, but this design has a number of deficiencies. On the one hand, once the single-tube containers are broken, the internal detection reagents are prone to leakage, which may cause reagent waste, and may also result in ambient contamination. On the other hand, it is difficult to guarantee the sealing of the single-tube containers, and reagent failure may be caused after long-time storage of the containers. In addition, the single-tube containers is not convenient and fast to operate, which cannot meet the requirement for rapid detection.

### SUMMARY

The embodiments aim to provide a rapid detection kit for pediatric respiratory infection, in order to solve the problems mentioned in the above background.

In order to achieve the above object, the present disclosure provides the following technical solution. A rapid detection kit for pediatric respiratory infection includes a first sub kit for containing a rapid detection reagent for pediatric respiratory infection. The first sub kit is externally sleeved with a first protective sleeve. A connecting ring is integrally formed on a the top end of the first protective sleeve. A second sub kit is threadedly connected inside the connecting ring. The second sub kit internally contains the rapid detection reagent for pediatric respiratory infection. A cap is threadedly connected to the top end of the second sub kit. The second sub kit is externally sleeved with a second protective sleeve. A bottom end of the second protective sleeve externally sleeves the connecting ring.

In some embodiments, the first protective sleeve is of a circular tube structure having a cross section in a shape of a Chinese character “⊏”. A bottom end of the first protective sleeve is fixed to an outer circumferential surface of the first sub kit. An inner circumferential diameter of the top end of the first protective sleeve is equal to an inner circumferential diameter of the first sub kit. An outer circumferential diameter of the connecting ring is less than an outer circumferential diameter of the first protective sleeve.

In some embodiments, the top end of the first protective sleeve is plugged with a seal cap The seal cap is of a circular frame structure in a shape of a Chinese character “ [symbol] ”. A bottom end of the seal cap is fixedly sleeved with a rubber stopper ring. The rubber stopper ring is a circular ring plate having an “L” shaped cross section. The rubber stopper ring is clamped between the seal cap and an inner circumferential opening of the first protective sleeve.

In some embodiments, an inner circumferential surface of the connecting ring is provided with a first internal thread. An outer circumferential surface of the bottom end of the second sub kit is provided with a first external thread. The first internal thread is fittingly connected to the first external thread.

In some embodiments, an inner circumferential surface of the bottom end of the second protective sleeve is provided with a first catch groove. The first catch groove is an annular groove, and an outer circumferential surface of the connecting ring is fixedly sleeved with a rubber ring. The bottom end of the second protective sleeve externally sleeves the connecting ring, and the rubber ring is inserted into the first catch groove.

In some embodiments, the second protective sleeve is of a circular tube structure having an “L”-shaped cross section. Each of an outer circumferential surface of the second protective sleeve and an outer circumferential surface of the first protective sleeve is provided with a finger groove. The finger groove is annular groove, and an outer circumferential diameter of the second protective sleeve is equal to an outer circumferential diameter of the first protective sleeve.

In some embodiments, an outer circumferential surface of the top end of the second sub kit is provided with a second external thread. An inner circumferential surface of the cap is provided with a second internal thread, and the second internal thread is connected with the second external thread in a fitted manner.

In some embodiments, a second catch groove is formed in a bottom end of the cap. The second catch groove is an annular groove with a breaking opening being a circular opening. The cap sleeves the top end of the second sub kit, a rubber sleeve is inserted into the second catch groove, the rubber sleeve sleeves a top end of a lead-in ring. A bottom end of the lead-in ring is fixed to a surface of the second sub kit. The rubber sleeve is of a circular ring structure having a circular cross section, and a diameter of a circumferential surface of the rubber sleeve is greater than a width of a notch of the second catch groove.

Compared with the prior art, the embodiments have the following beneficial effects.

The rapid detection kit for pediatric respiratory infection is designed with the first sub kit and the second sub kit which are respectively loaded the rapid detection reagent for pediatric respiratory infection, effectively increasing the loading capacity of the reagent. Furthermore, the designs of a seal cap and a rubber stopper ring are adopted, achieving the tight sealing of the tops of the sub kits, and avoiding the problems of leakage and failure of the reagent. The designs of the sub kits and the protective sleeves effectively avoid the problem of reagent leakage caused by the breakage of a single-tube container. Even if one sub kit is broken, the reagent in the other sub kit can still be used normally, such that reagent waste and ambient contamination are reduced.

### BRIEF DESCRIPTION OF THE DRAWINGS

FIG. 1 is a structural schematic diagram of the present disclosure;

FIG. 2 is a top view of a structure of the present disclosure;

FIG. 3 is a sectional view of the structure along line A-A in FIG. 2;

FIG. 4 is an enlarged schematic diagram of part A in FIG. 3;

FIG. 5 is an enlarged schematic diagram of the structure at part B in FIG. 3;

FIG. 6 is a schematic diagram of a connecting structure for a first sub kit and a connecting ring of the present disclosure;

FIG. 7 is a front view of the structure in FIG. 6;

FIG. 8 is a sectional view of a structure along line B-B in FIG. 7;

FIG. 9 is a structural schematic diagram of a connecting structure for a second sub kit and a second protective sleeve of the present disclosure;

FIG. 10 is a front view of the structure in FIG. 9;

FIG. 11 is a sectional view of a structure along line C-C in FIG. 10.

List of the reference characters: 1 first sub kit; 2 first protective sleeve; 3 connecting ring; 4 seal cap; 5 rubber stopper ring; 6 second sub kit; 7 first internal thread; 8 first external thread; 9 second protective sleeve; 10 first catch groove; 11 rubber ring; 12 finger groove; 13 cap; 14 second catch groove; 15 lead-in ring; 16 rubber sleeve; 17 second internal thread; and 18 second external thread.

### DETAILED DESCRIPTION OF THE EMBODIMENTS

In order to describe the object, the technical solution and the advantages of the present disclosure clearly and completely, embodiments of the present disclosure will be further illustrated in detail below in conjunction with the drawings. It should be understood that specific embodiments described herein are a part of, but not all of, the embodiments of the present disclosure and are merely intended to explain the embodiments of the present disclosure, not to limit the embodiments of the present disclosure. All other embodiments obtained by those of ordinary skill in the art without creative efforts shall fall within the scope of protection of the present disclosure.

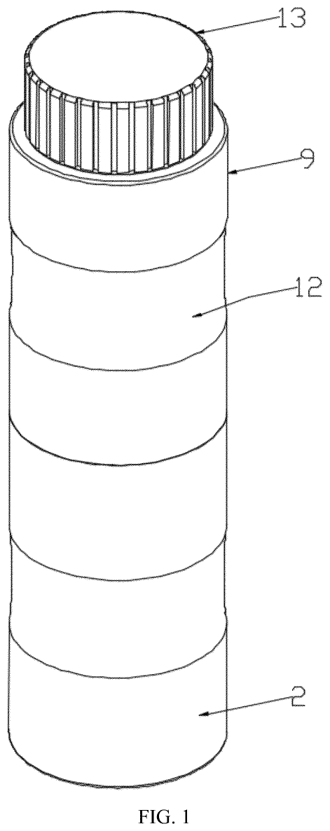

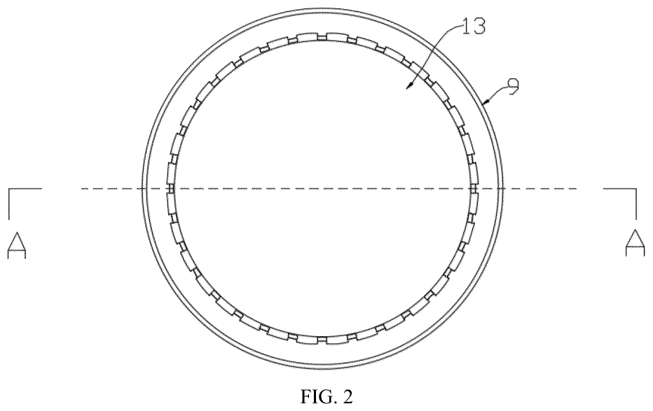

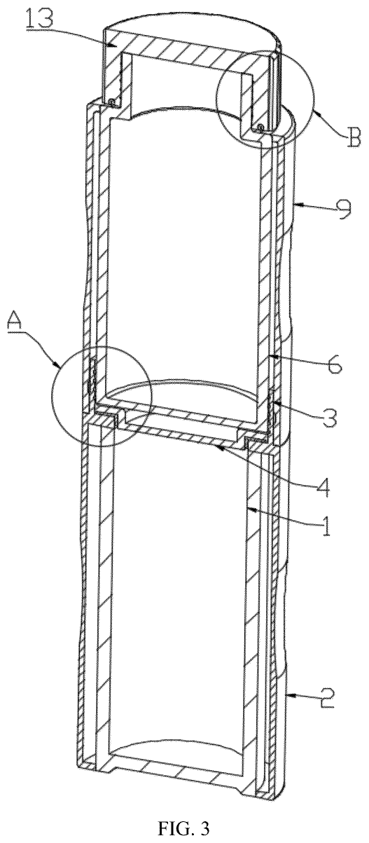

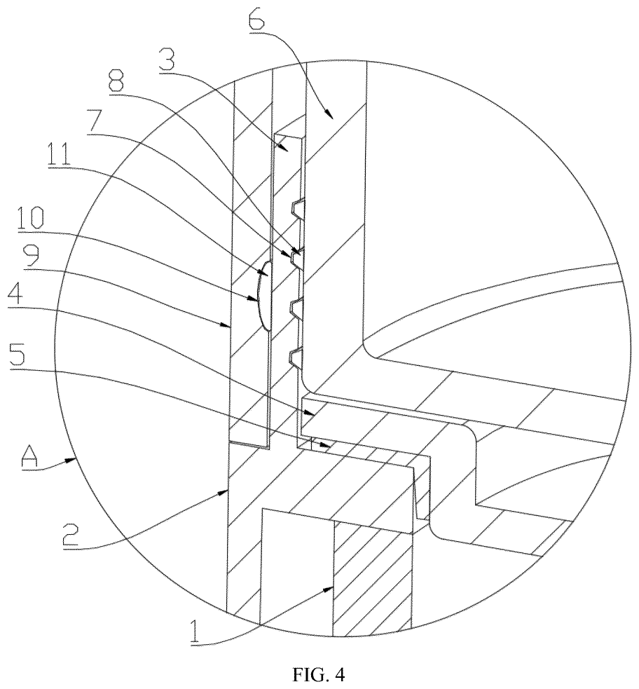

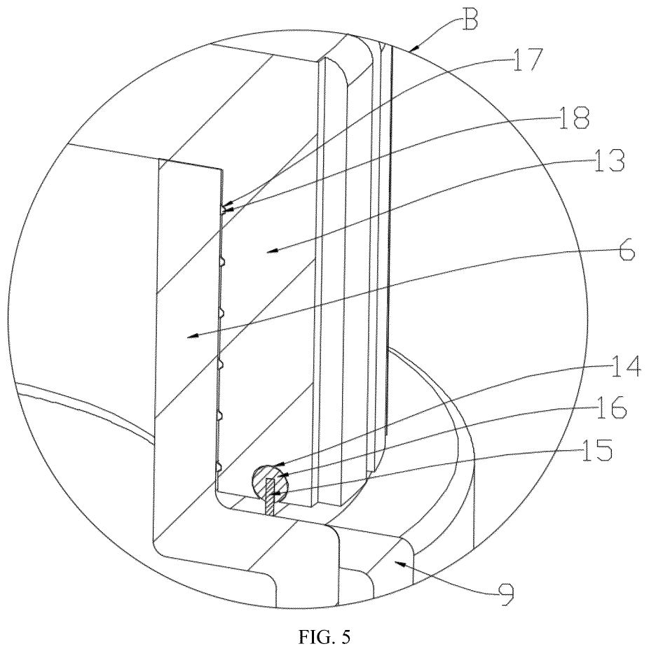

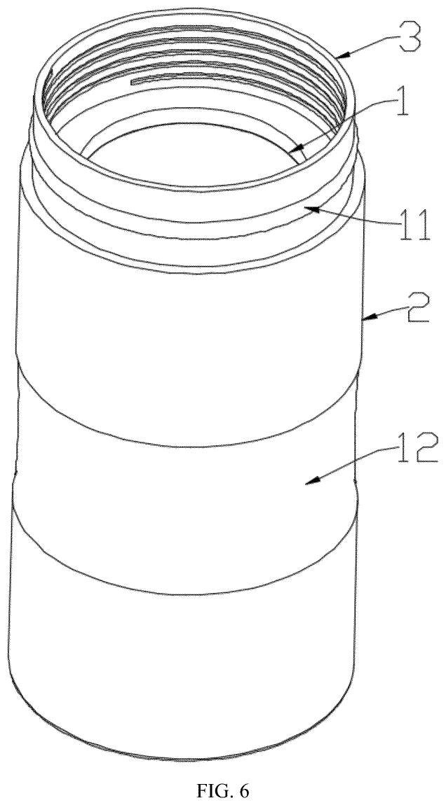

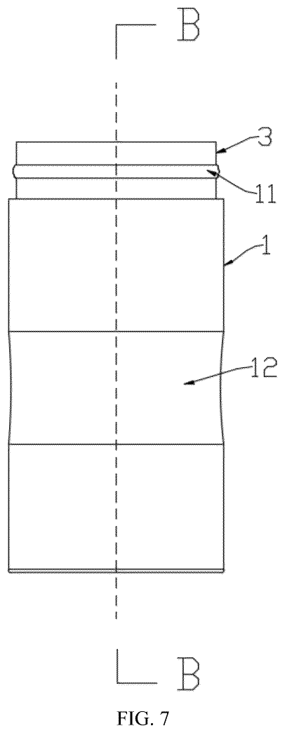

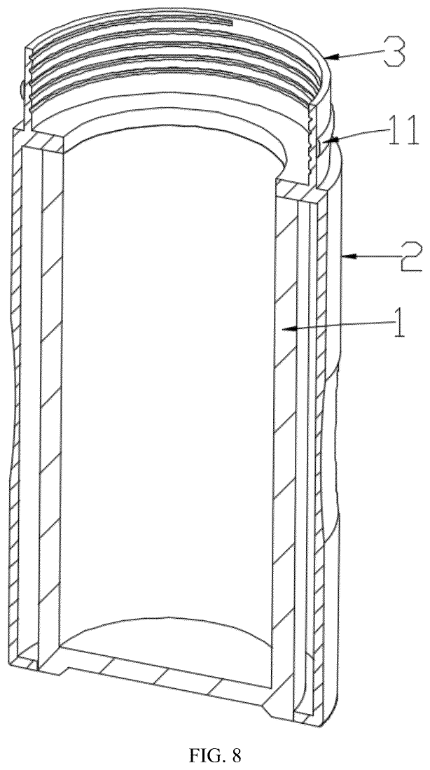

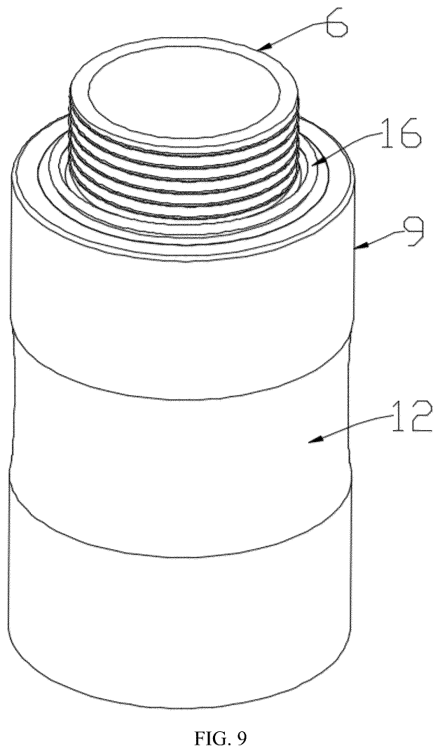

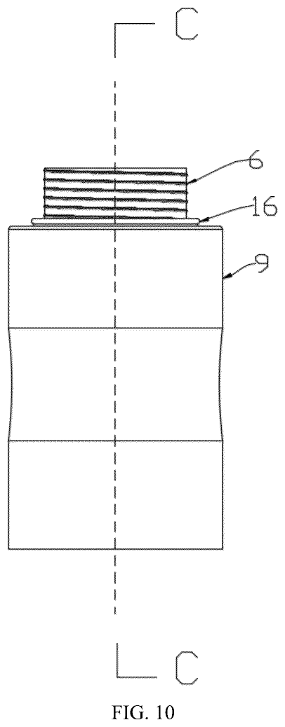

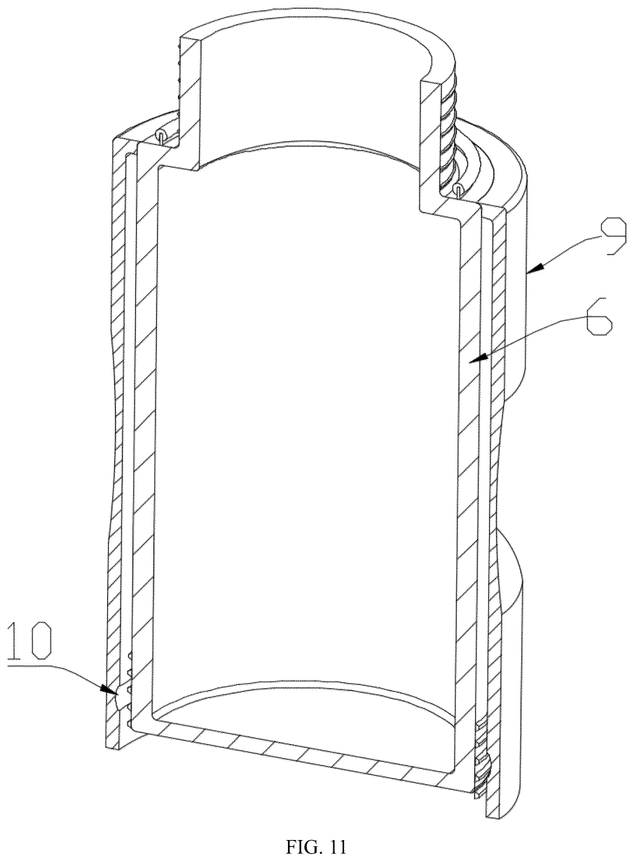

Referring to FIGS. 1-11, the present disclosure provides a technical solution as below. A rapid detection kit for pediatric respiratory infection includes a first sub kit 1 for containing a rapid detection reagent for pediatric respiratory infection. The fist sub kit 1 is externally sleeved with a first protective sleeve 2. The first protective sleeve 2 is of a circular tube structure having a cross section in a shape of a Chinese character “⊏”. The bottom end of the first protective sleeve 2 is fixed to an outer circumferential surface of the first sub kit 1. An inner circumferential diameter of the top end of the first protective sleeve 2 is equal to an inner circumferential diameter of the first sub kit 1. An outer circumferential diameter of a connecting ring 3 is less than an outer circumferential diameter of the first protective sleeve 2. The top end of the first protective sleeve 2 is plugged with a seal cap 4. The seal cap 4 is of a circular frame structure in a shape of a Chinese character “ [symbol] ”. The bottom end of the seal cap 4 is fixedly sleeved with a rubber stopper ring 5. The rubber stopper ring 5 is a circular ring plate having an “L” shaped cross section. The [symbol] stopper ring 5 is clamped between the seal cap 4 and an inner circumferential opening of the first protective sleeve 2. After the rapid detection reagent for pediatric respiratory infection is injected into the first sub kit 1, the seal cap 4 is inserted into a top notch of the first protective sleeve 2. At this time, the rubber stopper ring 5 is clamped between the seal cap 4 and the notch of the first protective sleeve 2 to achieve top sealing of the first sub kit 1.

The connecting ring 3 is integrally formed on the top end of the first protective sleeve 2 A second sub kit 6 is threadedly connected inside the connecting ring 3. The second sub kit 6 internally contains the rapid detection reagent for pediatric respiratory infection. An inner circumferential surface of the connecting ring 3 is provided with a first internal thread 7. An outer circumferential surface of the bottom end of the second sub kit 6 is provided with a first external thread 8. The first internal thread 7 is connected to the first external thread 8 in a fitted manner. A cap 13 is threadedly connected to the top end of the second sub kit 6 in a sleeved manner. The second sub kit 6 is externally sleeved with a second protective sleeve 9. The connecting ring 3 is externally sleeved with the bottom end of the second protective sleeve 9. An inner circumferential surface of the bottom end of the second protective sleeve 9 is provided with a first catch groove 10. The first catch groove 10 is an annular groove. An outer circumferential surface of the connecting ring 3 is fixedly sleeved with a rubber ring 11. After the bottom end of the second protective sleeve 9 externally sleeves the connecting ring 3, the rubber ring 11 is inserted into the first catch groove 10. The second protective sleeve 9 is of a circular tube structure having an “L” shaped cross section. Each of an outer circumferential surface of the second protective sleeve 9 and an outer circumferential surface of the first protective sleeve 2 is provided with finger grooves 12. The finger grooves 12 are annular grooves. An outer circumferential diameter of the second protective sleeve 9 is equal to the outer circumferential diameter of the first protective sleeve 2.

The bottom end of the second sub kit 6 is inserted and threadedly connected into an inner circumferential opening of the connecting ring 3. The bottom end of the second sub kit 6 presses down the seal cap 4 to prevent the seal cap 4 from jouncing at the top of the first sub kit 1. The second protective sleeve 9 externally sleeves the connecting ring 3. The rubber ring 11 is inserted into the first catch groove 10, and thus the firmness of the threaded connection between the second sub kit 6 and the connecting ring 3 is enhanced. When it is required to remove the second sub kit 6 from the top of the first sub kit 1, the finger groove 12 on a surface of the first protective sleeve 2 is pinched with the fingers of one hand, the finger groove 12 on a surface of the second protective sleeve 9 is pinched with the fingers of the other hand, and the second protective sleeve 9 is rotated to unscrew and remove the second sub kit 6 from the connecting ring 3. The first protective sleeve 2 and the second protective sleeve 9 respectively protect the first sub kit 1 and the second sub kit 6 to avoid the leakage of an internal liquid after the first sub kit 1 and the second sub kit 6 are damaged. The second sub kit 6 and the first sub kit 1 respectively contain the rapid detection reagent for pediatric respiratory infection to avoid the problem that a rapid detection reagent for pediatric respiratory infection leaks due to the breakage of a single-tube container and thus cannot be used.

An outer circumferential surface of the top end of the second sub kit 6 is provided with a second external thread 18. An inner circumferential surface of the cap 13 is provided with a second internal thread 17. The second internal thread 17 is connected with the second external thread 18 in a fitted manner. A second catch groove 14 is formed in the bottom end of the cap 13. The second catch groove 14 is an annular groove with a breaking opening being a circular opening. After the cap 13 sleeves the top end of the second sub kit 6, a rubber sleeve 16 is inserted into the second catch groove 14. The rubber sleeve 16 sleeves the top end of a lead-in ring 15. The bottom end of the lead-in ring 15 is fixed to a surface of the second sub kit 6. The rubber sleeve 16 is of a circular ring structure having a circular cross section, A diameter of a circumferential surface of the rubber sleeve 16 is greater than a width of a notch of the second catch groove 14. When the cap 13 is screwed to the top end of the second sub kit 6, as the cap 13 is lowered, the lead-in ring 15 and the rubber sleeve 16 are gradually pushed into the second catch groove 14. The rubber sleeve 16 functions as an elastic gasket after the cap 13 is threadedly connected to the second sub kit 6, and the rubber sleeve 16 is also snapped in the second catch groove 14 to play a downward pull role on the cap 13.

A detailed use procedure of the rapid detection kit for pediatric respiratory infection will be described below.

The first sub kit 1 is externally sleeved with the first protective sleeve 2 to ensure that the bottom end of the first protective sleeve 2 is fixed to the outer circumferential surface of the first sub kit 1. The connecting ring 3 is integrally formed on the top end of the first protective sleeve 2. The bottom end of the second sub kit 6 is inserted into the inner circumferential opening of the connecting ring 3, and the second sub kit is rotated and threadedly connected to ensure tight fit between the first internal thread 7 and the first external thread 8. The connecting ring 3 is externally sleeved with the second protective sleeve 9 to ensure that the rubber ring 11 is inserted into the first catch groove 10 of the bottom end of the second protective sleeve 9. It should be confirmed that the finger grooves 12 are clearly visible on the outer circumferential surfaces of the first protective sleeve 2 and the second protective sleeve 9, which is conducive to a subsequent operation.

When the first sub kit is loaded, the seal cap 4 is opened, and the rapid detection reagent for pediatric respiratory infection is injected into the first sub kit 1. One end of the seal cap 4 fixedly sleeved with the rubber stopper ring 5 is inserted into the top notch of the first protective sleeve 2. It should be ensured that the rubber stopper ring 5 is clamped between the seal cap 4 and the notch of the first protective sleeve 2 to achieve the top sealing of the first sub kit 1. When the second sub kit is loaded, the rapid detection reagent for pediatric respiratory infection is injected into the second sub kit 6. The cap 13 is screwed to the top end of the second sub kit 6 to ensure the tight fit of the second internal thread 17 and the second external thread 18. During screwing, it should be noted that the lead-in ring 15 and the rubber sleeve 16 are gradually pushed into the second catch groove 14 of the bottom end of the cap 13. It should be ensured that the rubber sleeve 16 snaps into the second catch groove 14 to play the downward pull role on the cap 13.

The rapid detection reagent for pediatric respiratory infection in the first sub kit 1 or the second sub kit 6 is selectively used according to actual requirements. If the first sub kit 1 is used, the seal cap 4 is opened directly. If the second sub kit 6 is used, the finger groove 12 on the surface of the first protective sleeve 2 is pinched with the fingers of one hand, the finger groove 12 on the surface of the second protective sleeve 9 is pinched with the fingers of the other hand, and the second protective sleeve 9 is rotated to unscrew and remove the second sub kit 6 from the connecting ring 3. As specified in the instructions for the rapid detection reagent of pediatric respiratory infection, the reagent in the sub kits is used.

Although embodiments of the present disclosure have been shown and described, it will be appreciated by those of ordinary skill in the art that various changes, modifications, substitutions, and variations may be made to the embodiments without departing from the principle and spirit of the present disclosure, and the scope of the present disclosure is defined by the appended claims and their equivalents.

What is claimed is:

1. A rapid detection kit for a pediatric respiratory infection, comprising a first sub kit for containing a rapid detection reagent for the pediatric respiratory infection, wherein the first sub kit is externally sleeved with a first protective sleeve, a connecting ring is integrally formed on a top end of the first protective sleeve, a second sub kit is threadedly connected inside the connecting ring, the second sub kit internally contains the rapid detection reagent for the pediatric respiratory infection, a cap is threadedly connected to a top end of the second sub kit, the second sub kit is externally sleeved with a second protective sleeve, and a bottom end of the second protective sleeve externally sleeves the connecting ring.

2. The rapid detection kit for the pediatric respiratory infection according to claim 1, wherein the first protective sleeve is of a circular tube structure having a cross section in a shape of a Chinese character “⊏”, a bottom end of the first protective sleeve is fixed to an outer circumferential surface of the first sub kit, an inner circumferential diameter of the top end of the first protective sleeve is equal to an inner circumferential diameter of the first sub kit, and an outer circumferential diameter of the connecting ring is less than an outer circumferential diameter of the first protective sleeve.

3. The rapid detection kit for the pediatric respiratory infection according to claim 1, wherein the top end of the first protective sleeve is plugged with a seal cap, the seal cap is of a circular frame structure in a shape of a Chinese character “ [symbol] ”, a bottom end of the seal cap is fixedly sleeved with a rubber stopper ring, the rubber stopper ring is a circular ring plate having an “L” shaped cross section, and the rubber stopper ring is clamped between the seal cap and an inner circumferential opening of the first protective sleeve.

4. The rapid detection kit for the pediatric respiratory infection according to claim 1, wherein an inner circumferential surface of the connecting ring is provided with a first internal thread, an outer circumferential surface of a bottom end of the second sub kit is provided with a first external thread, and the first internal thread is connected with the first external thread in a fitted manner.

5. The rapid detection kit for the pediatric respiratory infection according to claim 1, wherein an inner circumferential surface of the bottom end of the second protective sleeve is provided with a first catch groove, the first catch groove is an annular groove, and an outer circumferential surface of the connecting ring is fixedly sleeved with a rubber ring, the bottom end of the second protective sleeve externally sleeves the connecting ring, and the rubber ring is inserted into the first catch groove.

6. The rapid detection kit for the pediatric respiratory infection according to claim 1, wherein the second protective sleeve is of a circular tube structure having an “L”-shaped cross section, each of an outer circumferential surface of the second protective sleeve and an outer circumferential surface of the first protective sleeve is provided with a finger groove, the finger groove is an annular groove, and an outer circumferential diameter of the second protective sleeve is equal to an outer circumferential diameter of the first protective sleeve.

7. The rapid detection kit for the pediatric respiratory infection according to claim 1, wherein an outer circumferential surface of the top end of the second sub kit is provided with a second external thread, an inner circumferential surface of the cap is provided with a second internal thread, and the second internal thread is connected with the second external thread in a fitted manner.

8. The rapid detection kit for the pediatric respiratory infection according to claim 1, wherein a second catch groove is formed in a bottom end of the cap, the second catch groove is an annular groove with a breaking opening being a circular opening, the cap sleeves the top end of the second sub kit, a rubber sleeve is inserted into the second catch groove, the rubber sleeve sleeves a top end of a lead-in ring, a bottom end of the lead-in ring is fixed to a surface of the second sub kit, the rubber sleeve is of a circular ring structure having a circular cross section, and a diameter of a circumferential surface of the rubber sleeve is greater than a width of a notch of the second catch groove.
